(19) United States
(12) Patent Application Publication
(10) Pub. No.: US 2026/0263941 A1
(43) Pub. Date: Sep. 10, 2026

(54) REAL-TIME ESTIMATION OF USER ENGAGEMENT LEVEL AND OTHER FACTORS USING SENSORS

(71) Applicant: DOLBY LABORATORIES LICENSING CORPORATION

(72) Inventors: Andrea FANELLI, Seattle, WA (US); Nathan Carl SWEDLOW, Berkeley, CA (US); Evan David GITTERMAN, Palo Alto, CA (US); Jeffrey Ross BAKER, Thousand Oaks, CA (US); Scott DALY, Kalama, WA (US); Daniel Paul DARCY, San Francisco, CA (US); Alex BRANDMEYER, Berkeley, CA (US); Davis R. BARCH, San Mateo, CA (US)

(73) Assignee: DOLBY LABORATORIES LICENSING CORPORATION, (US)

(21) Appl. No.: 19/165,984

(22) PCT Filed: Mar. 19, 2024
(86) PCT No.: PCT/US2024/020537

§ 371 Date: Sep. 16, 2025

Related U.S. Application Data
(60) Provisional application No. 63/491,276, filed on Mar. 20, 2023.

(51) Int. Cl. A63F 13/67; A63F 13/212; A63F 13/213; G06F 3/01; G06T 11/60; H04S 7/00

(52) U.S. Cl. CPC A63F 13/67; G06F 3/013; G06T 11/60; H04S 7/302; A63F 13/212; A63F 13/213; H04S 2400/11

(57) ABSTRACT

Methods, systems, and devices involving estimating pupil dilation or constriction of a person viewing displayed media content due to cognitive load, arousal, or engagement. Some embodiments involve obtaining ambient illuminance data corresponding to illuminance of ambient light in the vicinity of a person, obtaining display screen luminance data associated with a content luminance value of the displayed media content and with display screen brightness, obtaining instantaneous pupil size data corresponding to a pupil size of one or more of the person's pupils, estimating a light-induced pupil dilation or constriction caused by the illuminance of ambient light and the luminance of a display screen being viewed by the person, and estimating the pupil dilation or constriction caused by at least one of engagement, arousal, or cognitive load experienced by the person based, at least in part, on the pupil size data and the light-induced pupil dilation or constriction.

### CROSS-REFERENCE TO RELATED APPLICATIONS

This application claims priority from U.S. Provisional Application Ser. No. 63/491,276 filed on Mar. 20, 2023, which is incorporated herein by reference.

### TECHNICAL FIELD

This disclosure pertains to devices, systems and methods for estimating user engagement levels and related factors based on signals from one or more sensors, as well as to responses to such estimated factors.

### BACKGROUND

Some methods, devices and systems for estimating user engagement, such as user engagement with advertising content, are known. Although existing devices, systems and methods can provide benefits in some contexts, improved devices, systems and methods would be desirable.

### SUMMARY

At least some aspects of the present disclosure may be implemented via one or more methods. In some instances, the method(s) may be implemented, at least in part, by a control system and/or via instructions (e.g., software) stored on one or more non-transitory media. Some methods involve estimating pupil dilation or constriction of a person viewing displayed media content due to cognitive load, arousal, or engagement.

Some such methods may involve obtaining, by a control system, ambient illuminance data corresponding to illuminance of ambient light in the vicinity of a person. Some such methods may involve obtaining, by the control system, instantaneous pupil size data corresponding to a pupil size of one or more of the person's pupils. According to some examples, the pupil size data may be obtained from a camera or an eye tracker. Some such methods may involve estimating, by the control system, based at least in part on the ambient illuminance data and the display screen luminance data, a light-induced pupil dilation or constriction caused by the illuminance of ambient light and the luminance of a display screen being viewed by the person. Some such methods may involve estimating, by the control system, the pupil dilation or constriction caused by at least one of engagement, arousal, or cognitive load experienced by the person based, at least in part, on the pupil size data and the light-induced pupil dilation or constriction.

In some examples, estimating the light-based pupil dilation or constriction may involve applying a pupil model to the illuminance of ambient light and the luminance of the display screen being viewed by the person. According to some examples, the pupil model may include personalized pupil model parameters based on measured responses of one or more of the person's pupils to luminance and illuminance. In some examples, determining the personalized pupil model parameters may involve estimating the light-based pupil dilation or constriction according to the pupil model, measuring the instantaneous pupil size and determining an estimation error based on a difference between a measured instantaneous pupil size and an estimated light-based pupil dilation or constriction according to the pupil model. According to some examples, the pupil model may be based, at least in part, on a cube of a cosine of a visual angle centered on a foveal position. In some examples, the pupil model may be also based, at least in part, on weightings of light wavelengths according to a luminosity function of vision.

Some methods may involve obtaining, by the control system, gaze direction data. In some examples, the content luminance data may be based, at least in part, on the gaze direction data.

Some methods may involve estimating, by the control system, a content time interval corresponding to an estimated pupil dilation caused by engagement or cognitive load. According to some examples, content corresponding to the content time interval may include video content, audio content, or a combination thereof. In some examples, estimating the content time interval corresponding to the pupil dilation caused by engagement or cognitive load may involve applying a time shift corresponding to a pupil dilation latency period. According to some examples, the time shift may be in a range from 1 to 3 seconds.

Some methods may involve estimating an engagement level, an arousal level, a cognitive load level, or combinations thereof based, at least in part, on an estimated pupil dilation or constriction caused by engagement, arousal or cognitive load. Some such methods may involve outputting analytics data based on an estimated engagement level, an estimated arousal level, an estimated cognitive load level, or combinations thereof.

Some methods may involve altering one or more aspects of the media content in response to the estimated pupil dilation or constriction caused by at least one of engagement, arousal or cognitive load. In some examples, the one or more aspects of the media content may be altered after a time during which the pupil dilation or constriction is estimated.

According to some examples, the displayed media content may be part of a video game. Altering the one or more aspects of the media content may include altering a difficulty level of the video game, generating one or more personalized gaming experiences based on engagement level, tracking the cognitive challenge of the player for competitive gaming, or combinations thereof. In some examples, generating the one or more personalized gaming experiences based on engagement level may involve environment modification, aesthetic modification, animation modification, game mechanic modification, or combinations thereof.

In some examples, the displayed media content may be part of an online learning course. Altering the one or more aspects of the media content may involve altering one or more aspects of the online learning course. According to some examples, altering the one or more aspects of the online learning course may involve altering an amount of information provided in the online learning course, altering an amount of time spent on at least a portion of the online learning course, altering a difficulty level of at least a portion of the online learning course, or combinations thereof.

According to some examples, altering the one or more aspects of the media content may involve modifying a distinguishability of a graphical object. In some examples, the graphical object may correspond to a person or a topic. According to some examples, modifying the distinguishability of the graphical object may involve altering a camera angle, modifying a time during which the graphical object is displayed, modifying a size in which the graphical object is displayed, or combinations thereof.

In some examples, altering the one or more aspects of the media content may involve altering one or more aspects of audio content. According to some examples, altering the one or more aspects of audio content may involve adaptively controlling an audio enhancement process. In some examples, altering the one or more aspects of audio content may involve altering one or more spatialization properties of the audio content. According to some examples, altering the one or more spatialization properties of the audio content may involve rendering at least one audio object at a different location than a location at which the at least one audio object would otherwise have been rendered.

Some or all of the operations, functions and/or methods described herein may be performed by one or more devices according to instructions (e.g., software) stored on one or more non-transitory media. Such non-transitory media may include memory devices such as those described herein, including but not limited to random access memory (RAM) devices, read-only memory (ROM) devices, etc. Accordingly, some innovative aspects of the subject matter described in this disclosure can be implemented via one or more non-transitory media having software stored thereon.

At least some aspects of the present disclosure may be implemented via apparatus. For example, one or more devices (e.g., a system that includes one or more devices) may be capable of performing, at least in part, the methods disclosed herein. In some implementations, an apparatus is, or includes, an audio processing system having an interface system and a control system. The control system may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system may be configured for implementing some or all of the methods disclosed herein.

Details of one or more implementations of the subject matter described in this specification are set forth in the accompanying drawings and the description below. Other features, aspects, and advantages will become apparent from the description, the drawings, and the claims. Note that the relative dimensions of the following figures may not be drawn to scale.

### BRIEF DESCRIPTION OF THE DRAWINGS

Like reference numbers and designations in the various drawings indicate like elements.

FIG. 1A is a block diagram that shows examples of components of an apparatus capable of implementing various aspects of this disclosure.

FIG. 1B is a system diagram that shows an environment that includes examples of system components capable of implementing various aspects of this disclosure.

FIG. 2 shows examples of relationships between sensor types, sensor output and derived user-related metrics.

FIG. 3 shows examples of factors that affect pupil size in humans.

FIG. 4 shows examples of blocks that may be involved in developing a pupil model corresponding to measured pupil responses of an individual person.

FIG. 5 shows example blocks of a process of estimating experience-based pupil dilation or contraction.

FIG. 6 is a flow diagram that outlines one example of a disclosed method.

### DETAILED DESCRIPTION OF EMBODIMENTS

We currently spend a lot of time consuming media content, including but not limited to audiovisual content, interacting with media content, or combinations thereof. (For the sakes of brevity and convenience, both consuming and interacting with media content may be referred to herein as “consuming” media content.) Consuming audiovisual content may involve viewing a television program, viewing a movie, gaming, video conferencing, participating in an online learning course, etc. Accordingly, movies, online games, video games, video conferences, online learning course, etc., may be referred to herein as types of audiovisual content. Other types of media content may include audio but not video, such as podcasts, streamed music, etc.

Previously-implemented approaches to estimating user engagement, etc., with media content such as movies, television programs, etc., do not take into account how a person reacts while the person is in the process of consuming the media content. Instead, a person's impressions may be assessed according to the person's rating of the content after the user has consumed it, such as after the person has finished watching a movie or an episode of a television program, after the user has played an online game, etc.

It would be beneficial to estimate one or more states of a person while the person is in the process of consuming media content. Such states may include, or may involve, user engagement, cognitive load, attention, interest, etc.

Various disclosed examples overcome the limitations of previously-implemented approaches to estimating user engagement. Some such examples involve using one or more cameras, eye trackers, ambient light sensors, microphones, wearable sensors, or combinations thereof. Some such examples involve measuring a person's level of engagement, heart rate, cognitive load, attention, interest, etc., while the person is consuming media content by watching a television, playing a game, participating in a telecommunication experience (such as a videoconference, a video seminar, etc.), listening to a podcast, etc. Some examples may involve altering one or more aspects of the media content in response to estimated engagement, arousal, cognitive load, etc.

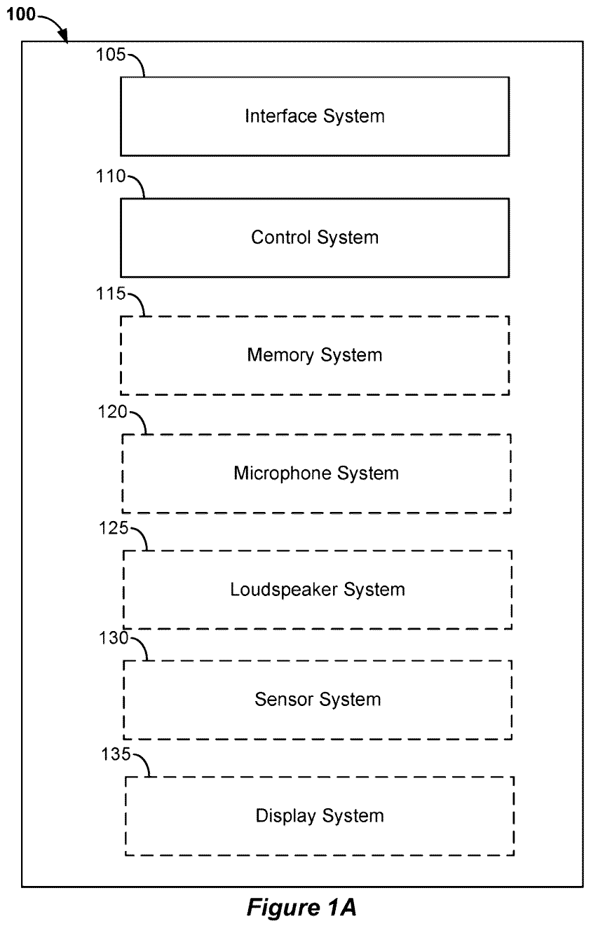

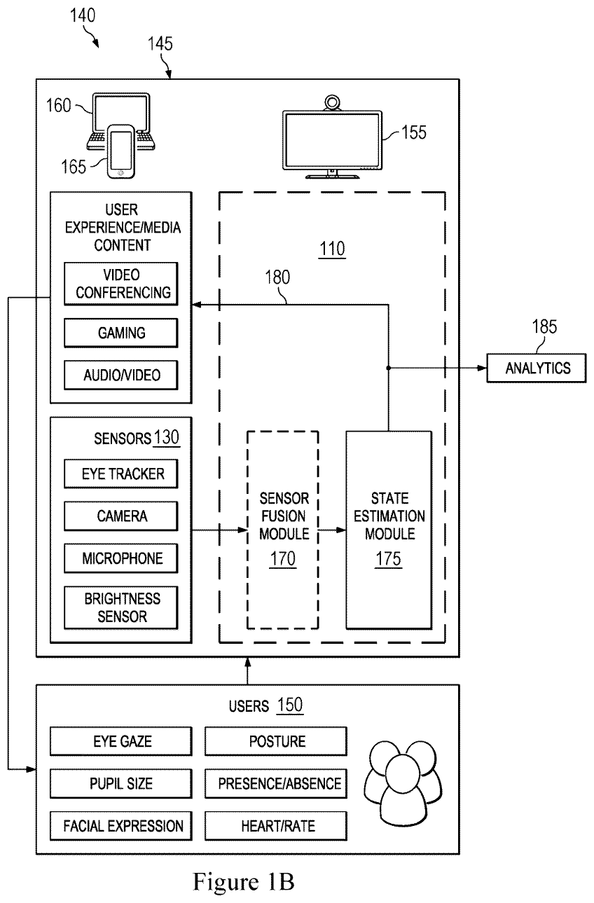

FIG. 1A is a block diagram that shows examples of components of an apparatus capable of implementing various aspects of this disclosure. As with other figures provided herein, the types, numbers and arrangements of elements shown in FIG. 1A are merely provided by way of example. Other implementations may include more, fewer and/or different types, numbers and arrangements of elements. According to some examples, the apparatus 100 may be configured for performing at least some of the methods disclosed herein. In some implementations, the apparatus 100 may be, or may include, one or more components of a workstation, one or more components of a home entertainment system, etc. For example, the apparatus 100 may be a laptop computer, a tablet device, a mobile device (such as a cellular telephone), an augmented reality (AR) wearable, a virtual reality (VR) wearable, an automotive subsystem (e.g., infotainment system, driver assistance or safety system, etc.), a game system or console, a smart home hub, a television or another type of device.

According to some alternative implementations the apparatus 100 may be, or may include, a server. In some such examples, the apparatus 100 may be, or may include, an encoder. In some examples, the apparatus 100 may be, or may include, a decoder. Accordingly, in some instances the apparatus 100 may be a device that is configured for use within an environment, such as a home environment, whereas in other instances the apparatus 100 may be a device that is configured for use in “the cloud,” e.g., a server.

According to some examples, the apparatus 100 may be, or may include, an orchestrating device that is configured to provide control signals to one or more other devices. In some examples, the control signals may be provided by the orchestrating device in order to coordinate aspects of displayed video content, of audio playback, or combinations thereof. In some examples, the apparatus 100 may be configured to alter one or more aspects of media content that is currently being provided by one or more devices in an environment in response to estimated user engagement, estimated user arousal or estimated user cognitive load. Some examples are disclosed herein.

In this example, the apparatus 100 includes an interface system 105 and a control system 110. The interface system 105 may, in some implementations, be configured for communication with one or more other devices of an environment. The environment may, in some examples, be a home environment. In other examples, the environment may be another type of environment, such as an office environment, an automobile environment, a train environment, a street or sidewalk environment, a park environment, an entertainment environment (e.g., a theatre, a performance venue, a theme park, a VR experience room, an e-games arena), etc. The interface system 105 may, in some implementations, be configured for exchanging control information and associated data with other devices of the environment. The control information and associated data may, in some examples, pertain to one or more software applications that the apparatus 100 is executing.

The interface system 105 may, in some implementations, be configured for receiving, or for providing, a content stream. In some examples, the content stream may include video data and audio data corresponding to the video data. The audio data may include, but may not be limited to, audio signals. In some instances, the audio data may include spatial data, such as channel data and/or spatial metadata. Metadata may, for example, have been provided by what may be referred to herein as an “encoder.”

The interface system 105 may include one or more network interfaces and/or one or more external device interfaces (such as one or more universal serial bus (USB) interfaces). According to some implementations, the interface system 105 may include one or more wireless interfaces. The interface system 105 may include one or more devices for implementing a user interface, such as one or more microphones, one or more speakers, a display system, a touch sensor system, a gesture sensor system, or combinations thereof. Accordingly, while some such devices are represented separately in FIG. 1A, such devices may, in some examples, correspond with aspects of the interface system 105.

In some examples, the interface system 105 may include one or more interfaces between the control system 110 and a memory system, such as the optional memory system 115 shown in FIG. 1A. Alternatively, or additionally, the control system 110 may include a memory system in some instances. The interface system 105 may, in some implementations, be configured for receiving input from one or more microphones in an environment.

The control system 110 may, for example, include a general purpose single- or multi-chip processor, a digital signal processor (DSP), an application specific integrated circuit (ASIC), a field programmable gate array (FPGA) or other programmable logic device, discrete gate or transistor logic, discrete hardware components, or combinations thereof.

In some implementations, the control system 110 may reside in more than one device. For example, in some implementations a portion of the control system 110 may reside in a device within one of the environments referred to herein and another portion of the control system 110 may reside in a device that is outside the environment, such as a server, a game console, a mobile device (such as a smartphone or a tablet computer), etc. In other examples, a portion of the control system 110 may reside in a device within one of the environments depicted herein and another portion of the control system 110 may reside in one or more other devices of the environment. For example, control system functionality may be shared by an orchestrating device (such as what may be referred to herein as a smart home hub) and one or more other devices of the environment. In other examples, a portion of the control system 110 may reside in a device that is implementing a cloud-based service, such as a server, and another portion of the control system 110 may reside in another device that is implementing the cloud-based service, such as another server, a memory device, etc. The interface system 105 also may, in some examples, reside in more than one device.

In some implementations, the control system 110 may be configured to perform, at least in part, the methods disclosed herein. According to some examples, the control system 110 may be configured to obtain ambient illuminance data corresponding to illuminance of ambient light in the vicinity of a person. For example, the control system 110 may be configured to obtain ambient illuminance data from one or more light sensors of the apparatus 100 or from one or more light sensors of another device in the vicinity of the person, such as a device within half a meter of the person, within a meter of the person, within 2 meters of the person, etc. According to some examples, the control system 110 may be configured to obtain display screen luminance data associated with a luminance value of the displayed media content. As used herein, the term “display screen luminance” encompasses both display screen brightness (for example, display screen brightness corresponding to a display device setting) and the relative luminosity of the displayed content itself, such as a portion of the displayed content at which a person is currently gazing.

In some examples, the control system 110 may be configured to obtain instantaneous pupil size data corresponding to a pupil size of one or more of the person's pupils. The “instantaneous pupil size data” may, for example, include data from one or more cameras, one or more eye tracking devices, etc., corresponding to the size of one or more of the person's pupils. According to some examples, the control system 110 may be configured to estimate, based at least in part on the ambient illuminance data and the display screen luminance data, a light-induced pupil dilation or constriction caused by the illuminance of ambient light and the luminance of the display screen being viewed by the person. In some examples, the control system 110 may be configured to estimate the pupil dilation or constriction caused by at least one of engagement, arousal, or cognitive load experienced by the person based, at least in part, on the pupil size data and the light-induced pupil dilation or constriction. Some examples of these estimation processes are described below.

Some or all of the methods described herein may be performed by one or more devices according to instructions (e.g., software) stored on one or more non-transitory media. Such non-transitory media may include memory devices such as those described herein, including but not limited to random access memory (RAM) devices, read-only memory (ROM) devices, etc. The one or more non-transitory media may, for example, reside in the optional memory system 115 shown in FIG. 1A and/or in the control system 110. Accordingly, various innovative aspects of the subject matter described in this disclosure can be implemented in one or more non-transitory media having software stored thereon. The software may, for example, include instructions for controlling at least one device to perform some or all of the methods disclosed herein. The software may, for example, be executable by one or more components of a control system such as the control system 110 of FIG. 1A.

In some examples, the apparatus 100 may include the optional microphone system 120 shown in FIG. 1A. The optional microphone system 120 may include one or more microphones. According to some examples, the optional microphone system 120 may include an array of microphones. In some examples, the array of microphones may be configured to determine direction of arrival (DOA) and/or time of arrival (TOA) information, e.g., according to instructions from the control system 110. The array of microphones may, in some instances, be configured for receive-side beamforming, e.g., according to instructions from the control system 110. In some implementations, one or more of the microphones may be part of, or associated with, another device, such as a speaker of the speaker system, a smart audio device, etc. In some examples, the apparatus 100 may not include a microphone system 120. However, in some such implementations the apparatus 100 may nonetheless be configured to receive microphone data for one or more microphones in an environment via the interface system 110. In some such implementations, a cloud-based implementation of the apparatus 100 may be configured to receive microphone data, or data corresponding to the microphone data, from one or more microphones in an environment via the interface system 110.

According to some implementations, the apparatus 100 may include the optional loudspeaker system 125 shown in FIG. 1A. The optional loudspeaker system 125 may include one or more loudspeakers, which also may be referred to herein as “speakers” or, more generally, as “audio reproduction transducers.” In some examples (e.g., cloud-based implementations), the apparatus 100 may not include a loudspeaker system 125.

In some implementations, the apparatus 100 may include the optional sensor system 130 shown in FIG. 1A. The optional sensor system 130 may include one or more touch sensors, gesture sensors, motion detectors, cameras, eye tracking devices, or combinations thereof. In some implementations, the one or more cameras may include one or more free-standing cameras. In some examples, one or more cameras, eye trackers, etc., of the optional sensor system 130 may reside in a television, a mobile phone, a smart speaker, a laptop, a game console or system, or combinations thereof. In some examples, the apparatus 100 may not include a sensor system 130. However, in some such implementations the apparatus 100 may nonetheless be configured to receive sensor data for one or more sensors (such as cameras, eye trackers, camera-equipped monitors, etc.) residing in or on other devices in an environment via the interface system 110.

In some implementations, the apparatus 100 may include the optional display system 135 shown in FIG. 1A. The optional display system 135 may include one or more displays, such as one or more light-emitting diode (LED) displays. In some instances, the optional display system 135 may include one or more organic light-emitting diode (OLED) displays. In some examples, the optional display system 135 may include one or more displays of a television, a laptop, a mobile device, a smart audio device, an automotive subsystem (e.g., infotainment system, driver assistance or safety system, etc.), or another type of device. In some examples wherein the apparatus 100 includes the display system 135, the sensor system 130 may include a touch sensor system and/or a gesture sensor system proximate one or more displays of the display system 135. According to some such implementations, the control system 110 may be configured for controlling the display system 135 to present one or more graphical user interfaces (GUIs).

According to some such examples the apparatus 100 may be, or may include, a smart audio device, such as a smart speaker. In some such implementations the apparatus 100 may be, or may include, a wakeword detector. For example, the apparatus 100 may be configured to implement (at least in part) a virtual assistant.

FIG. 1B is a system diagram that shows an environment that includes examples of system components capable of implementing various aspects of this disclosure. As with other figures provided herein, the types, numbers and arrangements of elements shown in FIG. 1B are merely provided by way of example. Other implementations may include more, fewer and/or different types, numbers and arrangements of elements. In this example, the environment 140 includes a system 145 and one or more people 150, which also may be referred to herein as “users” 150. According to this example, the system 145 includes one or more televisions (TVs) 155, one or more laptop computers 160, and one or more cellular telephones (“cell phones”) 165, each of which are instances of the apparatus 100 of FIG. 1A. In some examples, one or more of the TV(s) 155, laptop computer(s) 160 or cell phone(s) 165 may include cameras, for example deployed as camera-embedded monitors. The system 145 may be, or may include, one or more components of a home entertainment system, one or more components of an office workstation, etc., depending on the particular implementation.

In some examples, one or multiple users 150 may be viewing (for example, sitting in front of) a laptop 160, a TV 155, a computer monitor screen, a cell phone 165, etc., using a videoconferencing application. In other examples, one or multiple users 150 may be watching a movie, watching a television program, listening to music, or playing videogames.

According to this example, the system 145 includes an instance of the sensor system 130 that is described with reference to FIG. 1A. The sensor system 130 may have various types, numbers and arrangements of elements, depending on the particular implementation. In some examples, the sensor system 130 may include one or more cameras pointed at, and configured to obtain pupil size data regarding, one or more of the users 150. According to some examples, the sensor system 130 may include one or more eye-tracking devices configured to track the gaze of, and in some instances configured to obtain pupil size data regarding, one or more of the users 150. In some examples, the sensor system 130 may include one or more ambient luminosity sensors, which also may be referred to herein as brightness sensors. According to some examples, the sensor system 130 may include one or more microphones. In some examples, the sensor(s) of the sensor system 130 may reside in or on multiple locations of the environment 140, whereas in other examples, the sensor(s) of the sensor system 130 may reside in or on a single device of the environment 140.

The sensor(s) of the sensor system 130 may obtain information from one or more users 150, such as eye-gaze information (for example, direction of gaze information), pupil size information, facial expression information, posture information, information regarding the presence or absence of one or more users 150, where one or more users 150 are sitting, the number of users 150 in the environment 140, the heart rate of one or more users 150, etc. If the sensor system 130 includes at least one microphone, the ambient noise in the environment 140, the speech of one or more users 150 may be detected. If the sensor system 130 includes at least one brightness sensor, the luminosity of the environment 140 can be measured.

According to this example, the state estimation module 175 is configured to estimate one or more user states, environmental states, etc., based, at least in part, on sensor data from the sensor system 130. In some examples, the optional sensor fusion module 170, when present, may be configured to process and analyze sensor data obtained by the sensor system 130 using one or more sensor fusion algorithms and to provide sensor fusion data to the state estimation module 175. For example, a simple linear regression algorithm could be used to map input sensor data into a level of engagement and state of the user. More complex machine learning models, such as Support Vector Machines, could be used for the same purpose. Multi-layer perceptron and deep neural networks could also be used. The user state estimated by the state estimation module 175 may, in some examples, include one or more metrics that describe the user's mental state, emotional state, physiological state, or combinations thereof. For example, the state estimation module 175 may estimate a user's sentiment, emotion, engagement, preference, focus of attention, heart rate, or combinations thereof. In some examples, the state estimation module 175 may determine or estimate a user's presence in the environment 140, identify the user, estimate the user's location in the environment 140, estimate the number of users in the environment 140, or combinations thereof. According to some examples, the state estimation module 175 may estimate the environmental state, such as the ambient luminosity, the noise level, background chatter (speech by one or more users 150), or combinations thereof. In some examples, the state estimation module 175 may determine or estimate how many people are present in the environment 140, their ID(s), how far they are from a display screen that is presenting content, or combinations thereof, based at least in part on microphone signals.

In this example, the state estimation module 175 (and the sensor fusion module 170, when present) are implemented by an instance of the control system 110 that is described with reference to FIG. 1A. The control system 110 may, in some examples, reside in a device within the environment 140. As noted in the description of FIG. 1A, the control system 110 may, in some implementations, reside in more than one device. For example, in some implementations a portion of the control system 110 may reside in a device within the environment 140 and another portion of the control system 110 may reside in a device that is outside the environment, such as a server, a game console, a mobile device (such as a smartphone or a tablet computer), etc. In other examples, a portion of the control system 110 may reside in a device within the environment 140 and another portion of the control system 110 may reside in one or more other devices of the environment 140. For example, control system functionality may be shared by an orchestrating device (such as what may be referred to herein as a smart home hub) and one or more other devices of the environment. In other examples, at least a portion of the control system 110 may reside in a device that is implementing a cloud-based service, such as a server, and another portion of the control system 110 may reside in another device that is implementing the cloud-based service, such as another server, a memory device, etc.

The state estimations of the state estimation module 175 may be used in different ways, depending on the particular implementation of the system 145. In some examples, the state estimations of the state estimation module 175 may be used to dynamically modify and adapt the user experience, as suggested by the arrow 180. In one such example, a game may be made relatively more or relatively less challenging based on a user's state, such as the user's emotional response. In another example, a movie or a television program may be dynamically modified based on a user's state, for example in an attempt to induce a higher level of engagement. Additional examples are provided below.

Alternatively, or additionally, the state estimations of the state estimation module 175 may be used to generate analytics 185. For example, the analytics 185 may be generated during a marketing call to quantify the engagement of users during the call or the general sentiment (positive or negative). In some examples, the state estimations of the state estimation module 175 may be used to provide information to understand the overall outcome of a call. Additional examples are provided below.

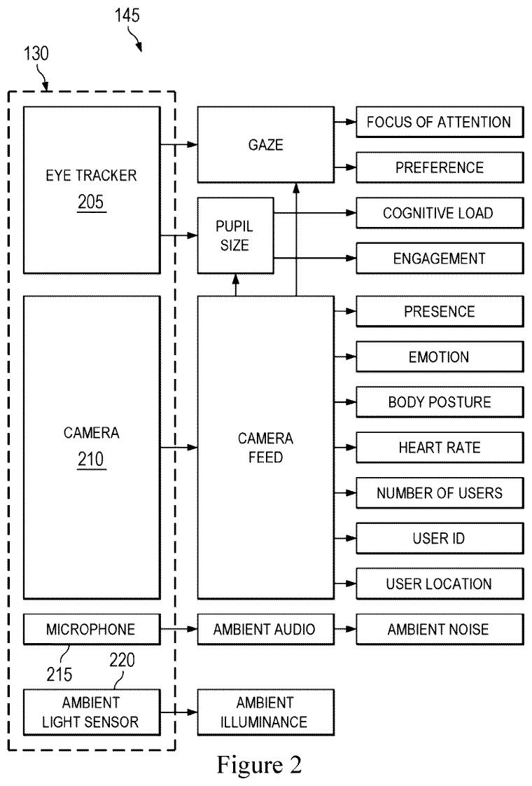

FIG. 2 shows examples of relationships between sensor types, sensor output and derived user-related metrics. As with other figures provided herein, the types, numbers and arrangements of elements shown in FIG. 2 are merely provided by way of example. Other implementations may include more, fewer and/or different types, numbers and arrangements of elements.

According to this example, the system 145 includes an instance of the sensor system 130 that is described with reference to FIG. 1A. The sensor system 130 depicted in FIG. 2 may, for example, reside in the environment 140 of FIG. 1B or in a similar environment. In this example, the sensor system 130 includes an eye-tracker 205, a camera 210, a microphone 215 and an ambient light sensor 220. In some examples, a single device (such as a TV, a computer monitor, a laptop computer, a cell phone, a game console, etc.) may include the entire sensor system 130 that is shown in FIG. 2. Alternatively, or additionally, one or more other devices residing in the same environment may include an instance of the sensor system 130.

In this example, the eye tracker 205 is configured to collect gaze and pupil size information. According to some examples, the eye tracker 205 also may be configured to provide information regarding the presence or absence of one or more users in at least a portion of an environment, such as in front of a display screen that is currently displaying media content. In some examples, the eye tracker 205 may be configured to provide information regarding the distance of one or more users from a display screen that is currently displaying media content. In some examples, the state estimation module 175 of FIG. 1B may use eye gaze information to quantify the focus of attention of a user and the user's preferences (for example, estimated by the amount of time that the user spends focusing on a displayed media content in general, the amount of time that the user spends focusing on a specific object, person, or region of interest of the displayed media content, etc.). The eye-gaze could also be used as an input system. For example, eye gaze information may be used to interact with one or more buttons in a graphical user interface (GUI), to control the camera view in games (for example, by re-orienting a virtual camera), etc.

Pupil size data may be obtained via the eye tracker 205, by the camera 210, or a combination thereof. Pupil size may be used to quantify factors such as user cognitive load and user engagement. Some examples of estimating factors such as user engagement and cognitive load from pupil size, pupil dilation and pupil constriction are described below.

The camera feed (which may include still images, video, or combinations thereof) obtained from the camera 210 can be used to quantify user presence, user emotion, body posture, etc. According to some examples, the state estimation module 175 may be configured to estimate a user's heart rate based on a video of the user's face. Relevant methods are disclosed in S. Sanyal and K. Nundy, Algorithms for Monitoring Heart Rate and Respiratory Rate From the Video of a User's Face (IEEE Journal of Translational Engineering in Health and Medicine 2018; 6:2700111), which is hereby incorporated by reference. In other examples, a heart rate may be estimated by a wearable device, such as a watch, a fitness tracking device or another type of wearable health monitoring device.

In some examples, the state estimation module 175 may be configured to quantify, based on the camera feed, how many people are in front of the camera and their location in the environment. In some examples, the state estimation module 175 may be configured to identify one or more users according to the camera feed, for example by implementing a facial recognition algorithm. User identification is relevant to providing personalized and tailored media consumption experiences.

The camera feed can also be used to estimate pupil size and eye gaze. Accordingly, it is not essential for the sensor system 130 to include an eye tracker. In some examples, the state estimation module 175 may be configured to estimate user distance, user engagement, cognitive load, or combinations thereof according to pupil size and eye gaze information obtained from the camera feed.

Microphone signals from the microphone 215 may be used to measure the noise level in the environment, the presence or absence of user speech, such as background chatter, etc. In some examples, the state estimation module 175 may be configured to enhance the audio experience based on such audio information, for example by increasing or decreasing the volume of audio corresponding to media content being presented in the environment according to ambient noise in the environment, by increasing or decreasing the volume of dialog in a media content stream, etc. Speech information recorded by the microphones can also be used as input to quantify user state and engagement. For example, voice loudness and inferred emotion based on speech information can be used to understand user's response to the experience, stress, and engagement level.

The ambient light sensor 220 may be used to measure the illuminance of ambient light in the vicinity of one or more people in the environment. Such ambient illuminance data may, for example, be used to establish a “baseline” pupil size corresponding with the illuminance of ambient light, e.g., when a person is not viewing and reacting to displayed content. Alternatively, or additionally, such ambient illuminance data may be used (for example, by the state estimation module 175 of FIG. 1B) to compensate for pupil dilation or constriction caused by changes in the illuminance of ambient light.

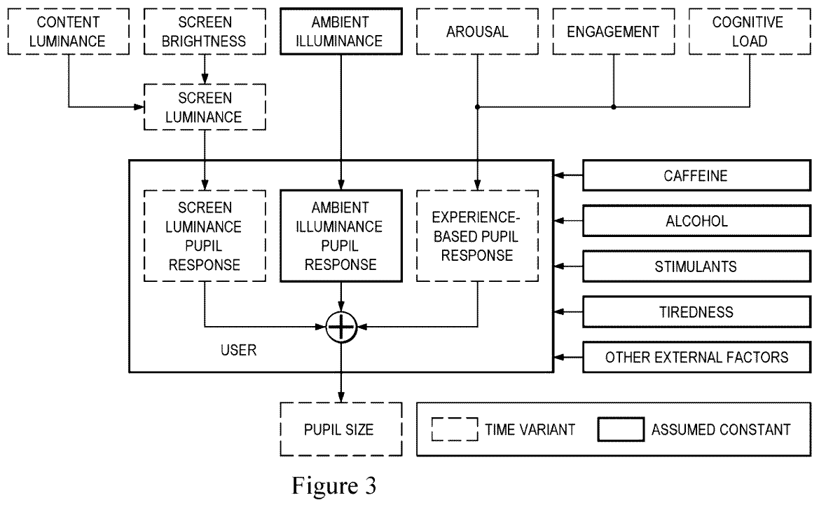

FIG. 3 shows examples of factors that affect pupil size in humans. The examples shown in FIG. 3 pertain to factors that directly contribute to pupil dilation and constriction in humans watching content on a screen. The brightness of the screen and the content brightness are components of display screen luminance (also referred to herein as “screen luminance”) and are major contributions to pupil size responses. Ambient illuminance also influences pupil size. Engagement, cognitive load, and arousal are other factors that affect pupil dilation and constriction. Factors such as engagement, cognitive load, and arousal may be referred to herein as “experience-based physiological responses” of a user, or simply as “experience-based responses” of a user. It is an underlying assumption of some disclosed implementations that, during times when a user is consuming media content, the emotional responses underlying the experience-based physiological responses of the user primarily- or entirely-correspond to the user's emotional responses to the media content being consumed.

According to some disclosed examples, pupil response may be considered to be the summation of these three main factors: screen luminance, ambient illuminance and the experience-based physiological responses of a user to media content being consumed. Some aspects of the present disclosure involve separating pupil responses caused by ambient illuminance and screen luminance from pupil responses caused by the experience-based physiological responses of a user.

The degrees to which screen luminance, ambient illuminance and experience-based physiological responses contribute to pupil dilation and constriction may vary substantially from person to person. Moreover, the degree to which the consumption of caffeine, alcohol, or other substances, as well as fatigue, affect pupil dilation or constriction (assuming a constant stimulus) also may vary substantially from person to person. We will refer to factors such as the consumption of caffeine, etc., herein as “user personal status.” User personal status factors may be considered to establish a baseline of an individual person's pupil size for the time and place of media content consumption.

In some examples, it will be assumed that the user personal status factors and the ambient illuminance are constant over the duration of a user's session of consuming media content. In other words, such examples involve an assumption that the ambient illuminance and the user personal status factors will remain constant during, for example, a video conferencing session, a movie watching session, or a gaming experience.

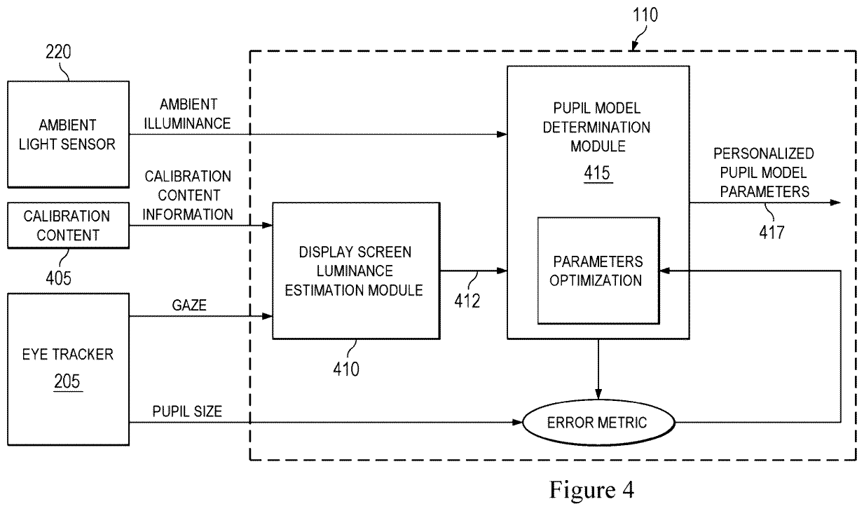

According to some disclosed examples, it will be assumed that if the ambient illuminance and the user personal status factors remain constant during a user's session of consuming media content, a person's pupil response will be based only on changes in display screen luminance and experience-based pupil responses. Therefore, some disclosed examples involve estimating experience-based pupil responses by subtracting estimated pupil responses based on display screen luminance changes. Some such examples involve obtaining data regarding an individual person's personalized pupil responses based on display screen luminance changes, for example via the process described herein with reference to FIG. 4, or by a similar process.

In other implementations, it will not be assumed that the ambient illuminance is constant during a user's session or experience of consuming media content. According to some such implementations, ambient illuminance data from an ambient light sensor may be used (for example, by the state estimation module 175 of FIG. 1B) to compensate for pupil dilation or constriction caused by changes in the illuminance of ambient light. Some such examples involve obtaining data regarding an individual person's personalized pupil responses based on changes in ambient light illuminance and using these data to compensate for pupil dilation or constriction caused by changes in the illuminance of ambient light.

Some disclosed examples involve developing what will be referred to herein as a “pupil model,” which may be a model of a particular person's light-induced pupil dilation or constriction caused by the luminance of a display screen being viewed by the person, a model of a particular person's light-induced pupil dilation or constriction caused by the illuminance of ambient light, or a combination thereof.

FIG. 4 shows examples of blocks that may be involved in developing a pupil model corresponding to measured pupil responses of an individual person. The pupil model may include personalized pupil model parameters based on measured responses of one or more of the person's pupils to luminance, measured responses of one or more of the person's pupils to illuminance, or to a combination thereof. The pupil model may be based on measurements of a current pupil size, which also may be referred to herein as an “instantaneous” pupil size, responsive to the luminance of a display screen being viewed by the person, responsive to the illuminance of ambient light, or a combination thereof.

According to the example shown in FIG. 4, an instance of the control system 110 of FIG. 1 is configured to implement the display screen luminance estimation module 410 and the pupil model determination module 415. According to this example, the pupil model determination module 415 is configured to determine the personalized pupil model parameters 417. In this example, determining the personalized pupil model parameters 417 involves an iterative process of estimating the light-induced pupil dilation or constriction according to current pupil model parameters, obtaining measurements of the instantaneous pupil size, determining an estimation error based on a difference between the measured instantaneous pupil size and an estimated light-based pupil dilation or constriction according to the pupil model parameters, and adjusting the current pupil model parameters according to the estimation error.

In the example shown in FIG. 4, the ambient light sensor 220 (also referred to herein as a brightness sensor) is configured to determine the ambient light illuminance in the vicinity of the person whose pupil responses are being evaluated (for example, within half a meter of the person, within a meter of the person, within 2 meters of the person, etc.) and to provide ambient illuminance data to the pupil model determination module 415.

Proper selection of the calibration content 405 is important for obtaining optimal pupil model parameters. The calibration content 405 may, in some examples, be identified to allow a stimulus that ranges from black to white, in other words from the minimum possible content luminance to the maximum possible content luminance.

According to this example, pupil size measurements are made with an eye tracker 205, which is configured to provide instantaneous pupil size data to the control system 110. In some alternative examples, instantaneous pupil size measurements may be made with a camera instead of, or in addition to, the eye tracker 205. In this example, the eye tracker 205 also determines a direction of the person's gaze and provides gaze information to the display screen luminance estimation module 410. In some examples the gaze information indicates, at the least, whether or not the person is viewing a display screen on which the calibration content 405 is being presented. In some examples, the gaze information also may indicate which portion of the display screen the person is viewing. As noted elsewhere herein, display screen luminance data may correspond both to the content luminance of the displayed media content—in this example, with the displayed calibration content 405—and with the brightness of the display screen on which the calibration content 405 is being presented.

Accordingly, based on gaze information that indicates which portion of the display screen the person is viewing and based on calibration content information, the display screen luminance estimation module 410 may, in some implementations, be configured to determine the content luminance of the calibration content that is being displayed in a particular area of the display screen. According to some examples, the display screen brightness may correspond with a brightness setting of the display screen. In some examples, the display screen luminance estimation module 410 is configured to determine the display screen luminance data 412 based on a content luminance value of the displayed calibration content and based on display screen brightness.

According to some examples, the display screen luminance estimation module 410 may be configured to determine the display screen luminance data 412 based, at least in part, on a “Y channel” portion of the calibration content information. The “Y channel” relates to the YUV color model, which takes human perception into account. The Y channel correlates approximately with perceived intensity, whereas the U and V channels provide color information. The display screen luminance estimation module 410 is configured to provide the display screen luminance data 412, however determined, to the pupil model determination module 415.

The pupil model determination module 415 may use different models to establish the relationship between input luminosity and output pupil size, depending on the particular implementation. Some such models are based in part on the assumption that the ambient illuminance has a generally lesser effect than the display screen luminance. This is generally true for viewing in a dim room, for example. The field of view (FOV) of the display is an important factor. In one simple example, the pupil model determination module 415 may apply a model that assumes the pupil size is controlled by a particular region (for example, a 4 degree region, a 6 degree region, an 8 degree region, a 10 degree region, etc.) around the fovea. The foveal position may, for example, be determined according to data from the eye tracker 205. According to some such examples, the model may also be based on an assumption that the pupil size is controlled solely by a particular component, such as the green component, of visible light. In other examples, the pupil model determination module 415 may apply a more nuanced model. Some such models involve using the cube of the cosine of the visual angle centered on the foveal position. Some such models also may involve weighting the wavelengths by a luminosity function of vision, such as the photopic luminous efficiency function established by the Commission Internationale de l′Éclairage (CIE). Some such models may take into account both the effect of the display screen luminance and the effect of the ambient illuminance, such as light reflecting from the walls surrounding the display. In some examples, the pupil model determination module 415 may apply a relatively more advanced model, such as a model based on a neural network, such as a recursive neural network (RNN), trained on large datasets of pupil size responsive to display screen luminance, ambient illuminance, or both. According to some such examples, at least a portion of the control system 110—such as the pupil model determination module 415—may be configured to implement the neural network. In some such examples, determining the error metric may involve applying a loss function that is used to train the neural network. According to some such examples, the estimation error may be, or may correspond to, a loss function gradient that is determined by applying the loss function.

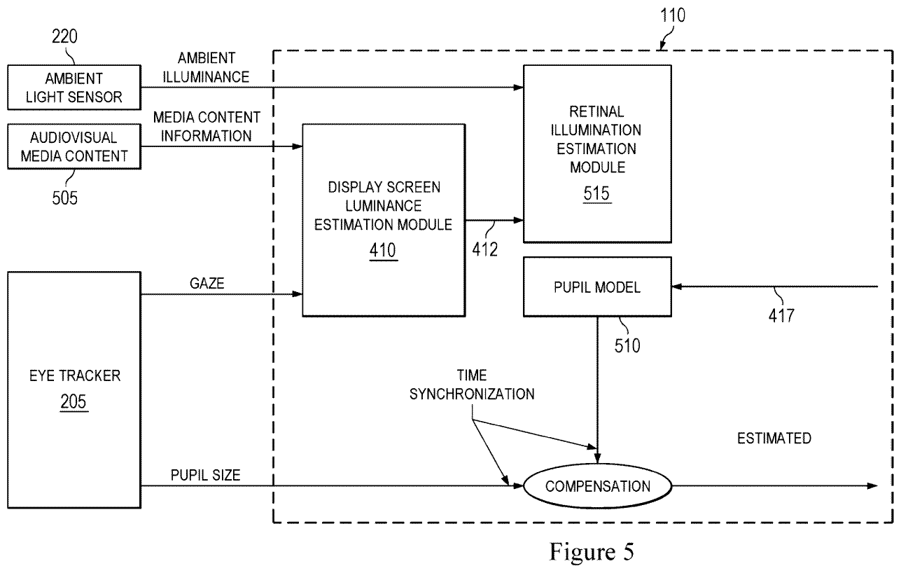

FIG. 5 shows example blocks of a process of estimating experience-based pupil dilation or contraction. As noted elsewhere herein, “experience-based pupil dilation or contraction” refers to pupil dilation or contraction caused by engagement, arousal, cognitive load, etc., experienced by a person responsive to an experience, such as the experience of consuming media content.

According to the example shown in FIG. 5, an instance of the control system 110 of FIG. 1 is configured to implement the display screen luminance estimation module 410 and the retinal illumination estimation module 515. According to this example, control system 110 is configured to make an estimation of light-induced pupil dilation or constriction by applying a pupil model 510 that is based, at least in part, on the personalized pupil model parameters 417. In some examples, the personalized pupil model parameters 417 may have previously been determined according to one or more of the processes described with reference to FIG. 4, or according to a similar process.

In the example shown in FIG. 5, the ambient light sensor 220 (also referred to herein as a brightness sensor) is configured to determine the ambient light illuminance in the vicinity of the person whose pupil responses are being evaluated (for example, within half a meter of the person, within a meter of the person, within 2 meters of the person, etc.) and to provide ambient illuminance data to the retinal illumination estimation module 515.

According to this example, pupil size measurements are made with an eye tracker 205, which is configured to provide instantaneous pupil size data to the control system 110. In some alternative examples, instantaneous pupil size measurements may be made with a camera instead of, or in addition to, the eye tracker 205. In this example, the eye tracker 205 also determines a direction of the person's gaze and provides gaze information to the display screen luminance estimation module 410.

In this example, the display screen luminance estimation module 410 is configured to determine the determine the display screen luminance data 412 according to one or more of the methods that are described with reference to FIG. 4. One small difference is that instead of receiving as input calibration content information corresponding to calibration content 405, the display screen luminance estimation module 410 receives media content information corresponding to the audiovisual media content 505 that is currently being viewed by the person whose pupil sizes are being evaluated.

In some examples, the control system 110 may be configured for estimating the experience-based pupil dilation or contraction by subtracting an estimated light-induced pupil dilation or contraction—here, estimated by the control system 110 according to the pupil model 510 and the personalized pupil model parameters 417—from the pupil dilation or contraction corresponding to the instantaneous pupil size measured by the eye tracker 205, measured by a camera, etc. According to some examples, the control system 110 may be configured for estimating the experience-based pupil dilation or contraction by subtracting the estimated light-induced pupil size from the instantaneous pupil size measured by the eye tracker 205, measured by a camera, etc. This is valid if we assume that the overall pupil dilation (or constriction) is the result of the addition (or subtraction) between the experience-based pupil-size change and the light-induced pupil-size change. The same reasoning can be applied if we assume that the two elements (light-induced pupil change and experience-based pupil change) contribute to overall pupil-size change through a multiplication of their effects. In that case, the experience-based pupil dilation (or constriction) would be obtained by dividing the overall pupil size change by the estimated light-induced pupil change.

As suggested by the “time synchronization” arrows of FIG. 5, there is generally a time lag between a displayed content time interval that causes an experience-based pupil dilation or contraction and the actual experience-based pupil dilation or contraction itself. This time may be referred to herein as a “pupil dilation latency period” and may, in some examples, be in the range of 1 to 3 seconds. Accordingly, some disclosed examples involve estimating at least the beginning of a content time interval corresponding to the pupil dilation caused by engagement or cognitive load by applying a time shift corresponding to a pupil dilation latency period. For example, if an experience-based pupil dilation or contraction is detected beginning at time T, some such examples may involve subtracting a time shift in the range of 1 to 3 seconds from time T in order to estimate the beginning of a time interval during which the displayed content produced the experience-based pupil dilation or contraction. The end of the time interval in which the displayed content produced the experience-based pupil dilation or contraction may, in some examples, be determined by subtracting the time shift from a time at which the experience-based pupil dilation or contraction ceases to occur.

Information regarding estimated experience-based pupil dilation or contraction may be used for many different applications, depending on the particular implementation. Following are some examples.

### Real Time Analytics

Some examples involve generating analytics regarding one or more users having a media content based experience such as watching a movie, watching a television program, videoconferencing, gaming, etc. Example of analytics that can be generated include estimates of sentiment/mood, attention, focus of attention, engagement, eye gaze, heart rate, etc. Some examples involve generating analytics regarding the presence or absence of a user, the number of users participating to the experience, their identification information, their location(s), the presence or absence of background chatter, etc.

According to some examples, the analytics may be generated for a single individual and provided (for example, displayed) in real time. Alternatively, or additionally, the analytics may be provided at the end of a single individual's media consumption experience. In some examples, the analytics may represent cumulative information, such as information regarding multiple instances of consuming media by a single individual.

Alternatively, or additionally, the analytics may be generated for a group of two or more people, for example if two or more people have been consuming the same media content. In some examples the analytics data may be anonymized, such that and only group analytics information is provided. Anonymized group analytics may be appropriate, for example, regarding a person's presentation to a large group, regarding a musician livestreaming to a crowd, etc. Other examples may involve generating post-teleconference analytics, such as for sales or marketing applications.

### Videoconferences

In case of videoconference experiences, the analytics may be used to improve the experience itself, for example according to a feedback loop. For example, eye gaze information may be used to modify a user interface (UI) and increase engagement. In case of certain videoconferencing experiences, the analytics may allow a participant (such as a presenter) to understand the level of the engagement of individual participants, of all participants in the aggregate, or combinations thereof. For example, the UI, the material presented, graphics, animations, etc., may be modified to encourage participants with low engagement levels to attend to presented material, to participate in a discussion, etc. In other embodiments, one or more aspects of the videoconference (such as windows corresponding to videos of the participants) may be modified in order to enhance and highlight people with relatively higher levels of engagement.

### Gaming

In some examples, the analytics may be generated while one or more people are playing videogames. In some gaming-related examples, analytics may quantify the performance of a player, the level of engagement of the player, the level of frustration of the player, the level of enjoyment of the player, the behavior of the player, etc. In some examples, the analytics may be used for user training, improving a user's gaming performance, etc. According to some examples, the difficulty or the design of the game may be dynamically adapted based on the analytics.

### Dialog Enhancement

In some examples, analytics may be used to quantify a user's level of challenge in understanding dialog. Some such examples may involve and adaptively controlling one or more dialogue enhancement features. Dialog enhancement may, for example, involve boosting the level of speech audio separately from the level of background content without increasing the overall loudness of the audio scene. According to some examples, analytics relating to dialog enhancement may include sentiment/emotion quantification, cognitive load estimated from pupil size, posture, facial expression, or combinations thereof.

### Personalized Video Content for TV and Streaming

By using sensors to understand the viewer's experience in real-time, there is untapped potential to personalize and customize the viewer's experience to suit the viewer's interest, preference, and mood. In some examples, video content may be automatically edited and enhanced to provide the best experience for a specific person at a specific time. Some such examples may involve using content metadata to specify options and intent.

For sports content, relevant analytics pertaining to a viewer's attention and engagement may include which player(s) the viewer is most interested in, which camera angles the viewer is most interested in, or both. With multiple camera feeds, such analytics may inform a switching/editing algorithm to show the user more of the player(s) the user is interested in. Even with a single camera feed, intelligent zoom or dynamic reframing techniques can be used to personalize the cropping. In some examples, custom overlays may be created and presented on the video content to show statistics or graphics that highlight the player(s) of interest. According to some examples, pre-existing cut scenes and graphics may also be customized in this manner.

For concert streams or previously recorded concerts, similar methods may be applied, such as switching between camera angles, zooming in on specific musicians of interest to a user, personalizing the audio mixing to focus on a vocalist or instrumentalist of particular interest, or combinations thereof.

There are many reality television shows with numerous actors or contestants, and often these shows will only air a small portion of the interviews and content that is filmed. By measuring the user's attention and engagement according to analytics such as those disclosed herein, an intelligent editing system may track which characters, actors, or contestants a user is most interested in, and then edit the content to show more of their interviews or content, in some instances including interviews or content that is not presented to most other viewers or listeners.

### Online Learning

According to some examples, one or more types of the disclosed analytics may be applied either in real-time or post-experience to improve online learning experiences. Online learning suffers from unique challenges that make it difficult for teachers to gauge students' engagement and comprehension, and difficult for students to stay focused and involved. However, online courses also offer unprecedented opportunities for personalizing the education experience. Because content is virtual and usually experienced alone, there is significant potential to customize the content to maximize the learning benefits for each individual student.

For example, estimated cognitive load may be used to classify how much effort an individual is exerting to retain or learn something. In some such examples, such analytics may be used to provide a more personalized learning experience. High cognitive load in the context of a learning process is not necessarily a negative factor. Some students may seek out challenges that cause a high cognitive load, whereas other students may quickly become discouraged and may even decide not to complete a course of instruction. Measuring and classifying different kinds of cognitive load signals can provide insights into whether a learner is motivated, disengaged, not retaining information, discouraged, distracted, etc. Some implementations may involve measuring complementary signals from a webcam, such as eye gaze, posture, facial expressions (furrowed brows, frowns, etc.) that can provide information about the student's experience. In some examples, UI metrics such as mouse movement, timing of button clicks, etc., may augment other types of analytics.

According to some examples, analytics may be aggregated and delivered in real-time to the teacher or presenter, so that the teacher or presenter can have a more interactive understanding of their audience's engagement, comprehension, etc. Such examples allow for a more interactive teaching or presenting experience.

In some examples, analytics may be provided as feedback to an educational software platform used for online learning. According to some examples, analytics may be used to intelligently personalize content so as to optimize engagement, retention, and conceptual understanding. In some examples, such modifications could be close to real-time: for example, more or less time could be spent on a particular topic, a lecture could be slowed down or sped up, additional context or information could be offered regarding particular topics, etc. In some examples, a different balance of learning modules, difficulty levels or content lengths may be suggested based on analytics data, such as analytics data gathered over multiple sessions.

According to some examples, at least some of the metrics collected may be presented directly to a user periodically. Such metrics may, for example, provide a student more insight into the student's learning patterns and may guide them in a positive way. Some students may enjoy having data about their learning progress and use it for motivation or positive reinforcement.

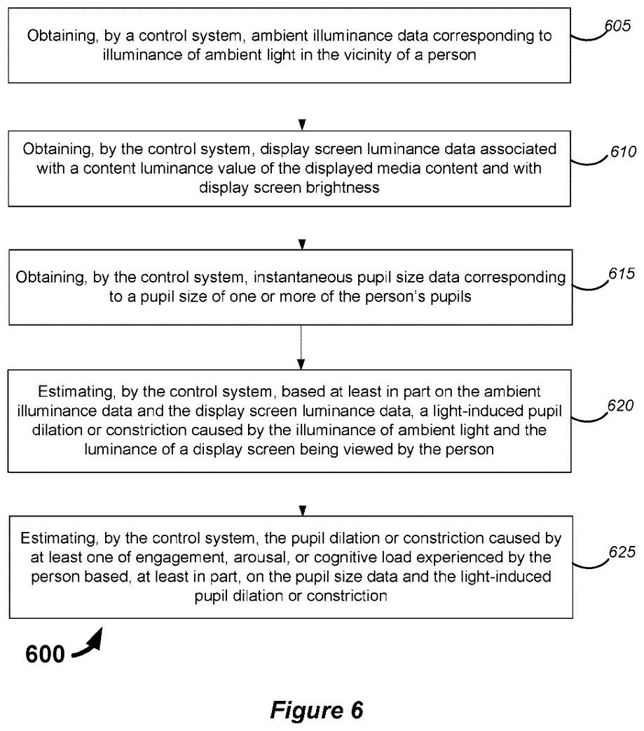

FIG. 6 is a flow diagram that outlines one example of a disclosed method. The blocks of method 600, like other methods described herein, are not necessarily performed in the order indicated. According to some examples, one or more blocks may be performed in parallel. Moreover, some similar methods may include more or fewer blocks than shown and/or described. In this example, method 600 involves estimating pupil dilation or constriction of a person viewing displayed media content due to cognitive load, arousal, engagement, or combinations thereof.

The method 600 may be performed by an apparatus or system, such as the apparatus 100 that is shown in FIG. 1A and described above. In some examples, the apparatus 100 includes at least the control system 110 shown in FIG. 5 and described above. In some examples, the blocks of method 600 may be performed by one or more devices within an audio environment, e.g., by an audio system controller (such as what may be referred to herein as a smart home hub) or by another component of an audio system, such as a television, a television control module, a laptop computer, a game console or system, a mobile device (such as a cellular telephone), etc. However, in some implementations at least some blocks of the method 600 may be performed by one or more devices that are configured to implement a cloud-based service, such as one or more servers.

In this example, block 605 involves obtaining, by a control system, ambient illuminance data corresponding to illuminance of ambient light in the vicinity of a person. In some examples, the control system may obtain the ambient illuminance data from a light sensor, such as the ambient light sensor 220 that is disclosed herein.

According to this example, block 610 involves obtaining, by the control system, display screen luminance data associated with a content luminance value of the displayed media content and associated with display screen brightness. According to some examples, block 610 may involve receiving display screen luminance data from the display screen luminance estimation module 410 of FIG. 5.

In this example, block 615 involves obtaining, by the control system, instantaneous pupil size data corresponding to a pupil size of one or more of the person's pupils. According to some examples, block 615 may involve receiving pupil size data from the eye tracker 205 of FIG. 5. In other examples, block 615 may involve receiving pupil size data from a camera.

According to this example, block 620 involves estimating, by the control system and based at least in part on the ambient illuminance data and the display screen luminance data, a light-induced pupil dilation or constriction caused by the illuminance of ambient light and the luminance of a display screen being viewed by the person. In some examples, block 620 may involve applying a pupil model and personalized pupil model parameters, such as described with reference to FIG. 5. The personalized pupil model parameters may, in some examples, be based on measured responses of one or more of the person's pupils to luminance and illuminance, such as described with reference to FIG. 4. Determining the personalized pupil model parameters may involve estimating the light-based pupil dilation or constriction according to the pupil model, measuring the instantaneous pupil size and determining an estimation error based on a difference between a measured instantaneous pupil size and an estimated light-based pupil dilation or constriction according to the pupil model (for example, as described above with reference to FIG. 4). In some examples, the pupil model may be based, at least in part, on a cube of a cosine of a visual angle centered on a foveal position. According to some examples, the pupil model may be based, at least in part, on weightings of light wavelengths according to a luminosity function of vision. In some examples, block 620 may involve estimating the light-based pupil dilation or constriction by applying the pupil model to the illuminance of ambient light and the luminance of the display screen being viewed by the person, to produce a light-induced pupil dilation or constriction estimate.

In this example, block 625 involves estimating, by the control system, the pupil dilation or constriction caused by at least one of engagement, arousal, or cognitive load experienced by the person based, at least in part, on the pupil size data and the light-induced pupil dilation or constriction. In some examples, block 625 may involve estimating the experience-based pupil dilation or contraction by subtracting the estimated light-induced pupil dilation or contraction from the pupil dilation or contraction corresponding to the instantaneous pupil size measured by the eye tracker 205, measured by a camera, etc. According to some examples, block 625 may involve estimating the experience-based pupil dilation or contraction by subtracting an estimated light-induced pupil size from an instantaneous pupil size measured by the eye tracker 205, measured by a camera, etc.

In some examples, method 600 may involve obtaining, by the control system, gaze direction data. In some such examples, the content luminance data may be based, at least in part, on the gaze direction data.

According to some examples, method 600 may involve estimating, by the control system, a content time interval corresponding to an estimated pupil dilation caused by engagement or cognitive load. The content corresponding to the content time interval may include video content, audio content, or a combination thereof. In some examples, estimating the content time interval corresponding to the pupil dilation caused by engagement or cognitive load may involve applying a time shift corresponding to a pupil dilation latency period. The time shift may, in some examples, be in a range from 1 to 3 seconds. In other examples, the time shift may be a longer time interval or a shorter time interval. According to some examples, a person's pupil dilation latency period may have previously been determined, for example as part of a calibration process like that described with reference to FIG. 4.

In some examples, method 600 may involve estimating an engagement level, an arousal level, a cognitive load level, or combinations thereof based, at least in part, on an estimated pupil dilation or constriction caused by the engagement, arousal or cognitive load. According to some examples, a person's actual engagement level, an arousal level, a cognitive load level, or combinations thereof may have previously been determined and may have previously been correlated with the estimated pupil dilation or constriction caused by the engagement, arousal or cognitive load, for example as part of a calibration process that involved obtaining feedback from the person regarding actual levels of engagement, arousal, cognitive load, or combinations thereof. According to some examples, method 600 may involve outputting analytics data based on an estimated engagement level, an estimated arousal level, an estimated cognitive load level, or combinations thereof.

According to some examples, method 600 may involve altering one or more aspects of the media content in response to the estimated pupil dilation or constriction caused by at least one of engagement, arousal or cognitive load. In some examples, the one or more aspects of the media content may be altered after a time during which the pupil dilation or constriction is estimated.

In some examples, the displayed media content may be part of a video game. Altering the one or more aspects of the media content may involve altering a difficulty level of the video game, generating one or more personalized gaming experiences (for example, based on engagement level), tracking the cognitive challenge of the player for competitive gaming, or combinations thereof. In some examples, generating the one or more personalized gaming experiences may involve environment modification, aesthetic modification, animation modification, game mechanic modification, or combinations thereof.

Whether in a gaming context or in another context (such as online learning, video conferencing, movie watching, etc.), according to some examples aesthetic modification may involve modifying a visual characteristic (for example, contrast, sharpness, mean lumiance level, color, hue, tone, color saturation, brightness, transparency, or other visual property) of a displayed graphical element (for example, a displayed graphical element of a game). In some examples, aesthetic modification may involve causing the addition to or removal of a graphical element from a plurality of displayed graphical elements. According to some examples, aesthetic modification may involve modifying an acoustic property of audio associated with a displayed graphical element (for example, changing a character's voice/accent/language, changing the volume, changing the background music, changing a notification sound, for example, a notification sound associated with a displayed chat message, etc.).

According to some examples, the displayed media content may be part of an online learning course. Altering the one or more aspects of the media content may involve altering one or more aspects of the online learning course. For example, altering the one or more aspects of the online learning course may involve altering an amount of information provided in the online learning course, altering an amount of time spent on at least a portion of the online learning course, altering a difficulty level of at least a portion of the online learning course, or combinations thereof.

In some examples, altering the one or more aspects of the media content may involve modifying a distinguishability of a graphical object. The graphical object may, for example, correspond to a person or a topic. Modifying the distinguishability of the graphical object may, for example, involve altering a camera angle, altering a viewing angle, modifying a time during which the graphical object is displayed, modifying a size in which the graphical object is displayed, or a combination thereof.

According to some examples, altering the one or more aspects of the media content may involve altering one or more aspects of audio content. In some examples, altering the one or more aspects of audio content may involve adaptively controlling an audio enhancement process, such as a dialogue enhancement process. According to some examples, altering the one or more aspects of audio content may involve altering one or more spatialization properties of the audio content. In some examples, altering one or more spatialization properties of the audio content may involve rendering at least one audio object at a different location than a location at which the at least one audio object would otherwise have been rendered.

Some aspects of present disclosure include a system or device configured (e.g., programmed) to perform one or more examples of the disclosed methods, and a tangible computer readable medium (e.g., a disc) which stores code for implementing one or more examples of the disclosed methods or steps thereof. For example, some disclosed systems can be or include a programmable general purpose processor, digital signal processor, or microprocessor, programmed with software or firmware and/or otherwise configured to perform any of a variety of operations on data, including an embodiment of disclosed methods or steps thereof. Such a general purpose processor may be or include a computer system including an input device, a memory, and a processing subsystem that is programmed (and/or otherwise configured) to perform one or more examples of the disclosed methods (or steps thereof) in response to data asserted thereto.

Some embodiments may be implemented as a configurable (e.g., programmable) digital signal processor (DSP) that is configured (e.g., programmed and otherwise configured) to perform required processing on audio signal(s), including performance of one or more examples of the disclosed methods. Alternatively, embodiments of the disclosed systems (or elements thereof) may be implemented as a general purpose processor (e.g., a personal computer (PC) or other computer system or microprocessor, which may include an input device and a memory) which is programmed with software or firmware and/or otherwise configured to perform any of a variety of operations including one or more examples of the disclosed methods. Alternatively, elements of some embodiments of the inventive system are implemented as a general purpose processor or DSP configured (e.g., programmed) to perform one or more examples of the disclosed methods, and the system also includes other elements (e.g., one or more loudspeakers and/or one or more microphones). A general purpose processor configured to perform one or more examples of the disclosed methods may be coupled to an input device (e.g., a mouse and/or a keyboard), a memory, and a display device.

Another aspect of the present disclosure is a computer readable medium (for example, a disc or other tangible storage medium) which stores code for performing (e.g., coder executable to perform) one or more examples of the disclosed methods or steps thereof.

While specific embodiments of the present disclosure and applications of the disclosure have been described herein, it will be apparent to those of ordinary skill in the art that many variations on the embodiments and applications described herein are possible without departing from the scope of the disclosure described and claimed herein. It should be understood that while certain forms of the disclosure have been shown and described, the disclosure is not to be limited to the specific embodiments described and shown or the specific methods described.

Various aspects of the present disclosure may be appreciated from the following Enumerated Example Embodiments (EEEs):

EEE1. A method of estimating pupil dilation or constriction of a person viewing displayed media content due to cognitive load, arousal, or engagement, the method comprising:
obtaining, by a control system, ambient illuminance data corresponding to illuminance of ambient light in the vicinity of a person;
obtaining, by the control system, display screen luminance data associated with a content luminance value of the displayed media content and with display screen brightness;
obtaining, by the control system, instantaneous pupil size data corresponding to a pupil size of one or more of the person's pupils;
estimating, by the control system, based at least in part on the ambient illuminance data and the display screen luminance data, a light-induced pupil dilation or constriction caused by the illuminance of ambient light and the luminance of a display screen being viewed by the person; and
estimating, by the control system, the pupil dilation or constriction caused by at least one of engagement, arousal, or cognitive load experienced by the person based, at least in part, on the pupil size data and the light-induced pupil dilation or constriction.
   EEE2. The method of EEE1, wherein estimating the light-based pupil dilation or constriction involves applying a pupil model to the illuminance of ambient light and the luminance of the display screen being viewed by the person.
   EEE3. The method of EEE2, wherein the pupil model includes personalized pupil model parameters based on measured responses of one or more of the person's pupils to luminance and illuminance.
   EEE4. The method of EEE3, wherein determining the personalized pupil model parameters involves estimating the light-based pupil dilation or constriction according to the pupil model, measuring the instantaneous pupil size and determining an estimation error based on a difference between a measured instantaneous pupil size and an estimated light-based pupil dilation or constriction according to the pupil model.
   EEE5. The method of EEE2, wherein the pupil model is based, at least in part, on a cube of a cosine of a visual angle centered on a foveal position.
   EEE6. The method of EEE5, wherein the pupil model is also based, at least in part, on weightings of light wavelengths according to a luminosity function of vision.
   EEE7. The method of any one of EEE1 to EEE6, wherein the pupil size data are obtained from a camera or an eye tracker.
   EEE8. The method of any one of EEE1 to EEE7, further comprising obtaining, by the control system, gaze direction data, wherein the content luminance data are based, at least in part, on the gaze direction data.
   EEE9. The method of any one of EEE1 to EEE8, further comprising estimating, by the control system, a content time interval corresponding to an estimated pupil dilation caused by engagement or cognitive load.
   EEE10. The method of EEE9, wherein content corresponding to the content time interval comprises video content, audio content, or a combination thereof.
   EEE11. The method of EEE9 or EEE10, wherein estimating the content time interval corresponding to the pupil dilation caused by engagement or cognitive load involves applying a time shift corresponding to a pupil dilation latency period.
   EEE12. The method of EEE11, wherein the time shift is in a range from 1 to 3 seconds.
   EEE13. The method of any one of EEE1 to EEE12, further comprising estimating an engagement level, an arousal level, a cognitive load level, or combinations thereof based, at least in part, on an estimated pupil dilation or constriction caused by engagement, arousal or cognitive load.
   EEE14. The method of EEE13, further comprising outputting analytics data based on an estimated engagement level, an estimated arousal level, an estimated cognitive load level, or combinations thereof.
   EEE15. The method of any one of EEE1 to EEE14, further comprising altering one or more aspects of the media content in response to the estimated pupil dilation or constriction caused by at least one of engagement, arousal or cognitive load.
   EEE16. The method of EEE15, wherein the one or more aspects of the media content are altered after a time during which the pupil dilation or constriction is estimated.
   EEE17. The method of EEE15 or EEE16, wherein the displayed media content is part of a video game and wherein altering the one or more aspects of the media content includes at least one of: altering a difficulty level of the video game; generating one or more personalized gaming experiences based on engagement level; or tracking the cognitive challenge of the player for competitive gaming.
   EEE18. The method of EEE17, wherein generating the one or more personalized gaming experiences based on engagement level involves at least one of environment modification, aesthetic modification, animation modification or game mechanic modification.
   EEE19. The method of EEE15, wherein the displayed media content is part of an online learning course and wherein altering the one or more aspects of the media content involves altering one or more aspects of the online learning course.
   EEE20. The method of EEE19, wherein altering the one or more aspects of the online learning course involves one or more of altering an amount of information provided in the online learning course, altering an amount of time spent on at least a portion of the online learning course or altering a difficulty level of at least a portion of the online learning course.
   EEE21. The method of EEE15, wherein altering the one or more aspects of the media content involves modifying a distinguishability of a graphical object.
   EEE22 The method of EEE21, wherein the graphical object corresponds to a person or a topic.
   EEE23. The method of EEE21 or EEE22, wherein modifying the distinguishability of the graphical object involves altering a camera angle, modifying a time during which the graphical object is displayed, modifying a size in which the graphical object is displayed, or a combination thereof.
   EEE24. The method of any one of EEE15 to EEE23, wherein altering the one or more aspects of the media content involves altering one or more aspects of audio content.
   EEE25. The method of EEE24, wherein altering the one or more aspects of audio content involves adaptively controlling an audio enhancement process.
   EEE26. The method of EEE24 or EEE25, wherein altering the one or more aspects of audio content involves altering one or more spatialization properties of the audio content.
   EEE27. The method of EEE26, wherein altering the one or more spatialization properties of the audio content involves rendering at least one audio object at a different location than a location at which the at least one audio object would otherwise have been rendered.
   EEE28. An apparatus configured to perform the method of any one of EEE1 to EEE27.
   EEE29. A system configured to perform the method of any one of EEE1 to EEE27.

1. A method of estimating pupil dilation or constriction of a person viewing displayed media content due to cognitive load, arousal, or engagement, the method comprising:
obtaining, by a control system, ambient illuminance data corresponding to illuminance of ambient light in the vicinity of a person;
obtaining, by the control system, display screen luminance data associated with a content luminance value of the displayed media content and with display screen brightness;
obtaining, by the control system, instantaneous pupil size data corresponding to a pupil size of one or more of the person's pupils;
estimating, by the control system, based at least in part on the ambient illuminance data and the display screen luminance data, a light-induced pupil dilation or constriction caused by the illuminance of ambient light and the luminance of a display screen being viewed by the person; and
estimating, by the control system, the pupil dilation or constriction caused by at least one of engagement, arousal, or cognitive load experienced by the person based, at least in part, on the pupil size data and the light-induced pupil dilation or constriction.

2. The method of claim 1, wherein estimating the light-based pupil dilation or constriction involves applying a pupil model to the illuminance of ambient light and the luminance of the display screen being viewed by the person.

3. The method of claim 2, wherein the pupil model includes personalized pupil model parameters based on measured responses of one or more of the person's pupils to luminance and illuminance.

4. The method of claim 3, wherein determining the personalized pupil model parameters involves estimating the light-based pupil dilation or constriction according to the pupil model, measuring the instantaneous pupil size and determining an estimation error based on a difference between a measured instantaneous pupil size and an estimated light-based pupil dilation or constriction according to the pupil model.

5. The method of claim 2, wherein the pupil model is based, at least in part, on a cube of a cosine of a visual angle centered on a foveal position.

6. (canceled)

7. The method of claim 1, wherein the pupil size data are obtained from a camera or an eye tracker.

8. The method of claim 1, further comprising obtaining, by the control system, gaze direction data, wherein the content luminance data are based, at least in part, on the gaze direction data.

9. The method of claim 1, further comprising estimating, by the control system, a content time interval corresponding to an estimated pupil dilation caused by engagement or cognitive load.

10. The method of claim 9, wherein content corresponding to the content time interval comprises video content, audio content, or a combination thereof.

11. The method of claim 9, wherein estimating the content time interval corresponding to the pupil dilation caused by engagement or cognitive load involves applying a time shift corresponding to a pupil dilation latency period.

12. (canceled)

13. The method of claim 1, further comprising estimating an engagement level, an arousal level, a cognitive load level, or combinations thereof based, at least in part, on an estimated pupil dilation or constriction caused by engagement, arousal or cognitive load.

14. (canceled)

15. The method of claim 1, further comprising altering one or more aspects of the media content in response to the estimated pupil dilation or constriction caused by at least one of engagement, arousal or cognitive load.

16. (canceled)

17. The method of claim 15, wherein the displayed media content is part of a video game and wherein altering the one or more aspects of the media content includes at least one of:
altering a difficulty level of the video game;
generating one or more personalized gaming experiences based on engagement level; or
tracking the cognitive challenge of the player for competitive gaming.

18. The method of claim 17, wherein generating the one or more personalized gaming experiences based on engagement level involves at least one of environment modification, aesthetic modification, animation modification or game mechanic modification.

19. (canceled)

20. (canceled)

21. The method of claim 15, wherein altering the one or more aspects of the media content involves modifying a distinguishability of a graphical object.

22. (canceled)

23. The method of claim 21, wherein modifying the distinguishability of the graphical object involves altering a camera angle, modifying a time during which the graphical object is displayed, modifying a size in which the graphical object is displayed, or a combination thereof.

24. The method of claim 15, wherein altering the one or more aspects of the media content involves altering one or more aspects of audio content.

25. (canceled)

26. The method of claim 24, wherein altering the one or more aspects of audio content involves altering one or more spatialization properties of the audio content.

27. (canceled)

28. An electronic device comprising:
one or more processors; and
memory storing one or more programs configured to executed by the one or more processors, the one or more programs including instructions for:
obtaining ambient illuminance data corresponding to illuminance of ambient light in the vicinity of a person;
obtaining display screen luminance data associated with a content luminance value of displayed media content and with display screen brightness;
obtaining instantaneous pupil size data corresponding to a pupil size of one or more of the person's pupils;
estimating, based at least in part on the ambient illuminance data and the display screen luminance data, a light-induced pupil dilation or constriction caused by the illuminance of ambient light and the luminance of a display screen being viewed by the person; and
estimating the pupil dilation or constriction caused by at least one of engagement, arousal, or cognitive load experienced by the person based, at least in part, on the pupil size data and the light-induced pupil dilation or constriction.

29. A non-transitory computer-readable medium storing one or more programs configured to be executed by one or more processors of an electronic device, the one or more programs including instructions for:
obtaining ambient illuminance data corresponding to illuminance of ambient light in the vicinity of a person;
obtaining display screen luminance data associated with a content luminance value of displayed media content and with display screen brightness;
obtaining instantaneous pupil size data corresponding to a pupil size of one or more of the person's pupils;
estimating, based at least in part on the ambient illuminance data and the display screen luminance data, a light-induced pupil dilation or constriction caused by the illuminance of ambient light and the luminance of a display screen being viewed by the person; and
estimating the pupil dilation or constriction caused by at least one of engagement, arousal, or cognitive load experienced by the person based, at least in part, on the pupil size data and the light-induced pupil dilation or constriction.
